(19) United States
(12) Patent Application Publication
(10) Pub. No.: US 2025/0041757 A1
(43) Pub. Date: Feb. 6, 2025

(54) PROCESS FOR PRODUCING METHANOL WITH UTILIZATION OF THE WASTE HEAT FROM AN ELECTROLYSER

(71) Applicant: L'Air Liquide, Societe Anonyme pour l'Etude et l’Exploitation des Procedes Georges Claude, Paris (FR)

(72) Inventors: Sebastian KOENIG, Frankfurt am Main (DE); Vincent PENA, Frankfurt am Main (DE)

(73) Assignee: L'Air Liquide, Societe Anonyme pour l'Etude et l’Exploitation des Procedes Georges Claude, Paris (FR)

(21) Appl. No.: 18/790,862

(22) Filed: Jul. 31, 2024

(30) Foreign Application Priority Data
Aug. 1, 2023 (EP) 23188953

(51) Int. Cl. B01D 3/00; C07C 29/152; C25B 9/67; C25B 15/08

(52) U.S. Cl. CPC B01D 3/007; C07C 29/152; C25B 9/67; C25B 15/081

(57) ABSTRACT

A process for preparing methanol on the basis of a synthesis gas stream having a hydrogen component and a carbon oxide component, wherein at least one of the components is produced at least partly in an electrolyser. The synthesis gas stream is converted to a crude methanol stream. Methanol is separated thermally from the crude methanol stream by distillations. Waste heat generated in the electrolyser is transferred to a circulating heat pump medium of a heat pump system, which results in at least partial evaporation of the heat pump medium, then the heat pump medium is compressed, and the heat released by the heat pump medium via at least partial condensation is transferred to a liquid medium by indirect heat transfer, and the heat transferred from the heat pump system to the liquid medium is utilized at least partly for the evaporation of the methanol-containing stream in the distillation evaporator.

### CROSS-REFERENCE TO RELATED APPLICATIONS

This application claims the benefit of priority under 35 U.S.C. § 119 (a) and (b) to EP patent application No. EP 23188953, filed Aug. 1, 2023, the entire contents of which are incorporated herein by reference.

### BACKGROUND

### Field of the Invention

The invention relates to a process for producing methanol from a synthesis gas stream, wherein the synthesis gas stream comprises a hydrogen component and a carbon dioxide component. It is a feature of the process that at least one of the components is provided at least partly by an electrolyser, and waste heat generated in the electrolyser is utilized by a heat pump system for the thermal separation of methanol from the crude methanol obtained in the methanol synthesis by distillation.

### Related Art

Production plants for the production of methanol comprise a distillation region for thermal separation of the crude methanol obtained in the methanol synthesis to methanol, water and by-products. For this purpose, heat at a temperature level of more than 90° C., preferably of more than 120° C., or of more than 150° C. is required.

Some of this heat can be provided in the form of mid-pressure steam, which is produced by boiler feed water in water-cooled methanol reactors on the shell side in the cooling of the catalyst bed. This mid-pressure steam can be expanded and saturated outside the reactor to a saturated steam pressure corresponding to the temperature required for distillation. However, this steam covers only a portion of the heat demand of the distillation; the remaining heat demand has to be provided in some other way.

Conventional methanol plants based on fossil fuels (for example natural gas) utilize high-temperature heat of frequently more than 300° C. from synthesis gas production, which is utilized in the form of steam, in order to provide the missing heat for the methanol distillation. Examples of synthesis gas production from fossil fuels are steam reforming of methane (SMR), autothermal reforming (ATR), partial oxidation (POx) and catalytic partial oxidation (CPO). The steam formed in synthesis gas production can likewise be expanded and saturated to a saturated steam pressure corresponding to the temperature required for distillation.

Different conditions exist in methanol production processes not based on the use of fossil fuels for synthesis gas production. For example, in what is called carbon dioxide-based methanol synthesis, a carbon dioxide stream, for example from a carbon dioxide separation plant, is preferably reacted with electrolytically produced hydrogen. The synthesis gas “production” in this case is thus merely the combination of the aforementioned streams. There is only a minor degree of occurrence, if any, of waste heat at a high temperature level that can be utilized as steam. Since the synthesis gas thus produced also contains barely any or no carbon monoxide, the actual methanol formation reaction proceeds with lower formation of heat (exothermicity) than would be the case if synthesis gas having a high carbon monoxide content were utilized. Thus, on the shell side of the water-cooled reactor in question, less steam is generated than would be the case in conventional methanol syntheses.

The aforementioned processes thus have a lack of utilizable heat from the outset.

As a result, heat has to be imported from a source outside the plant in question. This is usually accomplished in the form of steam that may originate from a supply grid in the vicinity, an electrical steam boiler, or recovery of heat within the plant that produces the carbon dioxide stream. Examples of plants that produce the carbon dioxide stream are power plants, cement works, steelworks and chemical production plants.

The importation of heat in the form of steam has the following disadvantages and problems, particularly with regard to methanol plants:
The steam has to be available in sufficient volume and under suitable conditions (for example at pressures of 2 to 6.5 bar; temperatures of more than 90° C., more than 120° C., or more than 150° C.; saturated or superheated) in the vicinity of the methanol plant.
The carbon dioxide footprint of the steam used should not worsen the carbon dioxide footprint of a carbon dioxide-based methanol plant, which is frequently the case—for example when the imported steam is produced by waste heat from a combustion process and/or oxidation process.
In relation to the utilization of heat or steam, methanol distillation is able to compete with a carbon dioxide separation plant, which likewise requires steam depending on the technology used. One example of such a technology is the chemical absorption of carbon dioxide on amines. Thermal regeneration of the carbon dioxide-laden amine solution requires steam.
At particular sites, the only power available is from renewable energy sources (solar power, wind power). This renewable power therefore has to be converted to heat, for example with the aid of an electrical steam boiler. At such a site, methanol distillation would compete with an electrolysis plant for the renewable power and hence significantly increase the power consumption and capacity of the renewable power source.

Electrolysis plants in principle release large amounts of waste heat, but at such a low temperature level that no steam with a temperature at which a methanol distillation would be heatable can be generated on this basis. This form of waste heat is frequently referred to as low-grade heat. The waste heat from an electrolyser is therefore frequently either released into the atmosphere, or fed into a district heating grid with or without a low-temperature heat pump at temperatures of in particular less than 90° C.

Modern electrolysers require a complex cooling system in order to release the waste heat formed in the electrochemical water splitting process to the environment. This is effected, for example, by means of air coolers, cooling water-operated plate heat exchangers and/or cooling towers. The utilization of at least some of the waste heat from an electrolyser would therefore reduce the operating costs (OPEX) and capital costs (CAPEX), and the space required by the electrolysis plant, especially the cooling system thereof. This can be accomplished, for example, by savings of added water for cooling towers or of power for air coolers.

In this connection, U.S. Pat. No. 11,247,955 B2 discloses a process for producing methanol from a carbon dioxide stream and a hydrogen stream, in which the hydrogen stream is provided by electrolysis of water. The waste heat from the electrolysis system is utilized as heat source for a heat pump system, and the heat released by the heat pump system is transferred to steam, which is utilized, for example, in an evaporator for the distillation of the crude methanol produced.

The procedure described by U.S. Pat. No. 11,247,955 B2 has the disadvantage that the heat released by the heat pump system is transferred directly to steam. Steam as a gaseous medium is unfavourable for the absorption of heat since the volume-specific heat absorption capacity thereof is much lower than that of a comparable liquid. For example, steam at 5 bar has a specific heat capacity of 2.5 KJ/kg/K, while liquid water at 5 bar has a specific heat capacity of 4.3 KJ/kg/K, such that the amount of steam required to transfer a defined heat flow at a defined temperature differential would be higher than that of water. Moreover, a steam stream of comparable volume requires greater pipeline diameter than a liquid since steam has a density several times lower than water, for example 2.7 kg/m³ (steam) compared to 915 kg/m³ (water). Large pipeline diameters increase the costs of the pipeline in question. If the heat should be provided to the consumer in the form of steam, it is possible by means of a phase change of the heated liquid, once a heat pump medium has transferred the heat thereto, with a specific enthalpy change of, for example, about 2100 KJ/kg for water, to utilize a defined amount of waste heat with significantly lower mass flow rates or temperature differentials in the heat carrier medium than in the case of heat transfer from the heat pump medium to steam.

What is likewise disadvantageous about steam (which is dry, saturated or superheated by definition) as heat-absorbing medium is the significantly lower and hence poorer coefficient of heat transfer a between heat exchanger wall and medium by comparison with a liquid to be heated. Corresponding heat exchangers, in accordance with the equation Q=α*A*ΔT, assuming equal temperature differential ΔT between wall and liquid or gaseous medium, require a considerably higher area A for compensation for the lower a. The larger area is reflected directly in the expenditures for components.

An electrolyser operated on the basis of PEM technology under full load can quite possibly release several times the heat imports required for a methanol plant as waste heat, in accordance with the hydrogen production capacity of that electrolyser which is required for methanol synthesis. However, the problem, as described before, is that the waste heat is regularly available at temperatures of only 40° C. to 60° C. (low grade heat). The difference between this electrolyser waste heat temperature and the temperature required for methanol distillation of regularly above 120° C. was therefore to be overcome with maximum efficiency with use of a heat pump system.

### SUMMARY OF THE INVENTION

It is thus a general object of the present invention to at least partly overcome the disadvantages of the prior art.

In particular, it is an object of the present invention to utilize the waste heat from an electrolysis plant with the aid of a heat pump system with maximum efficiency in a distillation plant for the separation of methanol from a methanol-containing stream.

A contribution to at least partial achievement of at least one of the above objects is made by the independent claims. The dependent claims provide preferred embodiments which contribute to at least partial achievement of at least one of the objects. Preferred embodiments of constituents of one category according to the invention are, where relevant, likewise preferred for identically named or corresponding constituents of a respective other category according to the invention. The expressions “having”, “comprising” or “containing” etc. do not preclude the possible presence of further elements, ingredients etc. The indefinite article “a” does not preclude the possible presence of a plurality.

The aforementioned objects are at least partially achieved by a process for producing methanol, comprising the process steps of:
a) providing a synthesis gas stream comprising at least a hydrogen component and a carbon oxide component, with at least one of the components being provided at least partly by an electrolyser;
b) converting the synthesis gas stream over a methanol synthesis catalyst in a reactor arrangement to give a gaseous crude methanol stream;
c) producing a liquid crude methanol stream by condensing and separating the crude methanol stream obtained in step b);
d) thermally separating methanol from the liquid crude methanol stream in a distillation apparatus, where the distillation apparatus has at least one distillation column in which a methanol-containing stream is evaporated in a distillation evaporator,
characterized in that
waste heat generated in the electrolyser is transferred by indirect heat transfer to a circulating heat pump medium of a heat pump system, which results in at least partial evaporation of the heat pump medium, then the heat pump medium is compressed, and the heat released by the heat pump medium via at least partial condensation is transferred to a liquid medium by indirect heat transfer, and the heat transferred from the heat pump system to the liquid medium is utilized at least partly for the evaporation of the methanol-containing stream in the distillation evaporator in step d).

The methanol-containing stream is a stream which is at least partly in the liquid phase, where the liquid component of this stream is evaporated, preferably fully evaporated, in the distillation evaporator. In relation to the methanol-containing stream, it is immaterial whether the stream is partly or completely in the liquid phase. All that is crucial is that at least some of the heat released by the heat pump medium is utilized for the evaporation of the liquid component of the methanol-containing stream.

The distillation apparatus comprises one or more distillation columns. If two or more distillation columns are encompassed by the distillation apparatus, these are preferably in a series arrangement. Preferably, each of the distillation columns generates a top product and a bottom product, where the bottom product boils at lower pressure and/or at lower temperature than the top product.

The liquid crude methanol stream produced in step c) is treated in the distillation apparatus with the purpose of separating methanol from the crude methanol stream. The aim here is in particular to obtain a stream of pure methanol. In the distillation apparatus, moreover, water and by-products are separated from the crude methanol stream.

The methanol-containing stream is that stream which is evaporated in the distillation evaporator of a distillation column. The stream (feed stream) fed to a distillation column differs in its composition from the methanol-containing stream since the distillation evaporator in particular forms part of the bottom of the respective column. The feed stream to a distillation column may be referred to as methanol-containing feed stream.

In the case of the first of the distillation columns in series arrangement, or if only one distillation column is present, the methanol-containing feed stream corresponds to the liquid crude methanol stream in terms of its composition. The methanol-containing feed stream may be referred to here as first methanol-containing feed stream.

In one example, in a first distillation column, low-boiling by-products are separated by a first methanol-containing feed stream. The bottom product is fed to a second distillation column as second methanol-containing feed stream. The top product formed in this second distillation column is essentially pure methanol, while the bottom product contains essentially water, with a methanol content of up to one percent by volume. In order to obtain further methanol, the bottom product from the second distillation column can be fed to a further, third distillation column as third methanol-containing feed stream.

At least one of the distillation columns of the distillation apparatus may have a side draw for a methanol- and water-containing stream.

Examples of suitable heat pump media are water, methanol, ethanol, hydrocarbons, oxygenates, halogenated or part-halogenated hydrocarbons, or hydrofluoroolefins.

Compression of the heat pump medium increases its temperature. In one embodiment, the temperature of the heat pump medium can be increased further by additional heating, for example by an external heat source. The external heat source may be an electrical heating apparatus. After compression, the heat pump medium releases heat to a liquid medium via indirect heat transfer, as a result of which the heat pump medium is at least partly condensed. In accordance with the principle of function of a compression heat pump, the pressure of the heat pump medium is lowered after the transfer of heat to the liquid medium. In one embodiment, this is accomplished by means of an expansion valve. Subsequently, the cycle starts again, i.e. the heat pump medium absorbs waste heat from the electrolyser, as a result of which it is at least partly evaporated.

Heat recovery and heat transfer via the heat pump medium in combination with the use of a liquid as medium to which the heat is transferred from the heat pump medium is an advantage over the approach taught in U.S. Pat. No. 11,247,955 B2. This proposes use of a medium in vapour form, to which the heat from the heat pump medium is transferred in a heat exchanger.

The heat transferred from the heat pump system to the liquid medium is utilized at least partly for the evaporation of the methanol-containing stream in the distillation evaporator according to step d). The fact that this heat is utilized for the evaporation of the methanol-containing stream does not necessarily mean that the methanol-containing stream is fully evaporated by the transfer of heat from the heat pump medium in the distillation evaporator. The transfer of heat from the heat pump medium increases the enthalpy of the methanol-containing stream in the distillation evaporator, but the methanol-containing stream can remain at least partly in the liquid state. Complete evaporation of the methanol-containing stream can be effected by means of a further downstream distillation evaporator, or one operated in parallel, or by means of a further heat exchanger structure in the same distillation evaporator via a further heating medium, for example steam.

Both the transfer of the waste heat from the electrolyser to the expanded heat pump medium and the transfer of the heat from the compressed heat pump medium to the liquid medium may be proximate or remote. In each case, however, the heat is transferred by indirect heat transfer, i.e. never directly from one medium to the other, which would result in mixing of the respective liquid or gaseous media.

Proximate heat transfer (1st option) in the context of the invention means that there is no further intervening auxiliary medium for the indirect transfer of heat. Proximate, indirect transfer of heat is effected, for example, in a heat exchanger from one medium to the other.

Remote heat transfer (2nd option) in the context of the invention means that the indirect transfer of heat is effected from the heat pump medium to an auxiliary medium, and then from the auxiliary medium to the liquid medium. Indirect transfer of heat from the heat pump medium to the auxiliary medium can be effected by means of a first heat exchanger. Indirect transfer of heat from the auxiliary medium to the liquid medium can be effected by means of a second heat exchanger. In a further option, the two steps can be combined within one heat exchanger that has two separate heat exchanger structures.

One advantage of the 1st option (proximate heat transfer) is inherently more efficient heat transfer via fewer and smaller heat exchangers because of the smaller number of heat carrier media used. A further advantage is the high temperature differentials in the heat exchangers, which result in lower flow rates of the heat pump medium within the heat pump circuit.

One advantage of the 2nd option (remote heat transfer) is an elevated safety level.

For example, in the event of a leak in a heat exchanger, no heat pump medium can get onto the process side of the electrolyser or of the methanol distillation plant. Auxiliary media for heat transfer can be chosen such that they do not impair the respective process side in the case of small leaks. Examples of suitable auxiliary media are water, water-glycol mixtures and thermal oil. Advantages also arise from the 2nd option in the case of maintenance of the heat pump:
The waste heat from the electrolyser can be more easily removed, for example by use of an air cooler that assists the cooling of the auxiliary medium;
The heat required by the distillation evaporator can be temporarily covered by the import of hot liquid or steam into the corresponding heat carrier circuit;
When boiling water is used as auxiliary medium, the auxiliary medium can be mixed with steam obtained on the shell side of a water-cooled methanol synthesis reactor. In one example, this is low-pressure steam.

The synthesis gas stream provided comprises at least a hydrogen component and a carbon oxide component. In other words, the synthesis gas stream comprises hydrogen and carbon monoxide and/or carbon dioxide.

At least one of the components is at least partly provided by an electrolyser. In particular, for the provision of the synthesis gas stream,
the hydrogen component can be at least partly produced electrolytically, or
the carbon oxide component can be at least partly produced electrolytically, especially carbon monoxide (CO) produced electrolytically,
or
the hydrogen component and the carbon oxide component can be at least partly produced electrolytically, especially hydrogen and carbon monoxide produced at least partly electrolytically.

In a preferred embodiment, the hydrogen component is provided completely by an electrolyser via electrolysis of a water-containing electrolysis medium. Examples of suitable methods are PEM electrolysis, alkaline electrolysis, AEM electrolysis, and high-temperature electrolysis with steam as electrolysis medium.

The process may also encompass several electrolysers, the waste heat from which is transferred by indirect heat transfer to a circulating heat pump medium of a heat pump system.

In a further preferred embodiment, the hydrogen component is provided at least partly by an electrolyser via electrolysis of a water-containing electrolysis medium, and the carbon oxide component in the form of a stream having a high carbon dioxide content. In one example, the stream having a high carbon dioxide content contains at least 50% by volume of carbon dioxide, preferably at least 75% by volume of carbon dioxide, further preferably at least 90% by volume of carbon dioxide, further preferably at least 95% by volume of carbon dioxide, further preferably at least 99% by volume of carbon dioxide, further preferably at least 99.5% by volume of carbon dioxide. This type of methanol synthesis is also referred to as CO2-based methanol synthesis. The stream having a high carbon dioxide content may come, for example, from a carbon dioxide separation plant, for example from an amine scrub, a physical scrub (e.g. methanol scrub), a membrane separation device, or a cryogenic carbon dioxide separation plant.

In a further embodiment, the carbon oxide component of the synthesis gas stream may be provided at least partly by electrolysis of a carbon dioxide-containing stream. Electrolysis of carbon dioxide reduces it at least partly to carbon monoxide. The resulting carbon monoxide-containing gas can be used as carbon oxide component for methanol synthesis.

The synthesis gas stream is converted in a reactor arrangement to a gaseous crude methanol stream over a suitable methanol synthesis catalyst, for example based on copper. The reactor arrangement may comprise one or more reactors connected in series and/or in parallel. In one embodiment, the reactor arrangement comprises two or more series-connected reactors, with intermediate condensation of the crude methanol stream downstream of each of the reactors, meaning that step c) is conducted downstream of each reactor of this reactor cascade. The reactors may in principle be water-cooled and/or gas-cooled reactors, irrespective of their arrangement.

The crude methanol stream contains at least methanol and water. In addition, it is regularly the case that unwanted by-products are present in the crude methanol stream, which can be removed as well in the thermal separation step d).

By condensation and separation of the gaseous crude methanol stream obtainable in step b), a liquid crude methanol stream is obtained in step c). Uncondensed constituents may, for example, be unconverted constituents of the synthesis gas stream, which, in the case of conventional methanol synthesis with a synthesis loop, are returned to the inlet of the reactor arrangement.

The thermal separation step d) serves in particular for separation of methanol from the liquid crude methanol stream. In particular, this affords at least one crude methanol stream, at least one water stream, and at least one by-product stream. For this purpose, a methanol-containing stream is evaporated in a distillation evaporator of at least one distillation column. The one or more distillation column(s) form(s) part of the distillation apparatus. The thermal separation in step d) may thus comprise two or more series-connected separation steps.

The heat introduced into the methanol-containing stream in the evaporation is transferred by the distillation process essentially to the top product from the corresponding distillation column, from which heat is withdrawn by a coolant. Examples of compositions of the methanol-containing stream are
a stabilized crude methanol, i.e. one that has been essentially freed of low-boiling components, in the bottom of a primary column as first distillation column and
a mixture with methanol and water as main components in the bottom of an atmospheric column as second distillation column with a lower methanol content than that in the crude methanol stream.

A preferred embodiment of the process according to the invention is characterized in that heat released by the heat pump medium is transferred proximately to the methanol-containing stream by indirect heat transfer in the distillation evaporator, where the methanol-containing stream comprises the liquid medium.

This preferred embodiment is an example of proximate transfer of heat released by the heat pump medium to a liquid medium. In this embodiment, the liquid medium is the methanol-containing stream, or is the liquid component of the methanol-containing stream which is evaporated in the distillation evaporator. In one embodiment, the methanol-containing stream is at least partly evaporated by the transfer of heat from the heat pump medium in the distillation evaporator.

In one embodiment, the methanol-containing stream is not completely evaporated by the transfer of heat from the heat pump medium in the distillation evaporator. In the latter embodiment, the methanol-containing stream, after partial evaporation by supply of heat via the heat pump medium, is then evaporated to completion (in series) by a further heat transfer medium, for example steam.

In a further embodiment, not the whole methanol-containing stream is evaporated by the transfer of heat from the heat pump medium in the distillation evaporator. In the latter embodiment, the fraction of the methanol-containing stream that has not been heated with the aid of the heat pump medium is evaporated by a further heat transfer medium, for example steam, in a further heat exchanger.

In this connection, it is immaterial that the heat transferred by the heat pump medium can in part also be transferred to gaseous crude methanol. What is crucial is that the heat provided by the heat pump medium is transferred at least partly to the methanol-containing stream, which is at least partly evaporated as a result.

The methanol-containing stream may be a liquid stream that has already been subjected to a distillation step. All that is crucial is that the methanol-containing stream contains methanol and water and/or by-products, such that a further distillation and hence evaporation in a distillation evaporator may be required in order to obtain a methanol stream having higher purity in relation to the methanol content of that stream, or else in order to obtain a water stream having higher purity in relation to the water content of that stream. The methanol-containing stream may be a stream having high methanol content and low water content. In that case, a further distillation may be required to obtain a methanol stream having higher purity in relation to the methanol content of that stream. The methanol-containing stream may also be a stream having high water content and low methanol content. In that case, a further distillation may be required to obtain a water stream having higher purity in relation to the water content of that stream.

A preferred embodiment of the process according to the invention is characterized in that heat released by the heat pump medium is transferred to a first auxiliary medium by indirect heat transfer, where the first auxiliary medium comprises the liquid medium, and heat absorbed by the first auxiliary medium is transferred to the methanol-containing stream by indirect heat transfer in the distillation evaporator.

The transfer of heat in the heat pump medium to the first auxiliary medium allows the latter either to remain liquid, or to at least partly evaporate as a result of the heat transfer. The statements made above are applicable to the transfer of heat from the first auxiliary medium in the distillation evaporator to the methanol-containing stream.

The methanol-containing stream need not necessarily be fully evaporated. All that is crucial in respect of this embodiment is that at least some of the heat in the heat pump medium is transferred to the first auxiliary medium, and at least some of this heat absorbed by the first auxiliary medium is transferred to the methanol-containing stream, such that this heat can be utilized for the evaporation of the methanol-containing stream. The heat absorbed by the first auxiliary medium can also be transferred to several methanol-containing streams in multiple distillation evaporators.

In one embodiment, the first auxiliary medium is water having a pressure of at least 3 bar, preferably of more than 5 bar, but not more than 90 bar. The water may either be heated and then routed to the distillation evaporator, or it may be evaporated in a pressure vessel in order to generate saturated steam. Steam generated in this way is then routed to the distillation evaporator. Optionally, the saturated steam stream thus generated can be compressed and saturated again, in order to increase the temperature and pressure thereof.

In a further embodiment, the first auxiliary medium is thermal oil.

A preferred embodiment of the process according to the invention is characterized in that the reactor arrangement includes a water-cooled reactor, and a steam stream is generated in a cooling system of the water-cooled reactor, and the steam stream is at least partly combined with the first auxiliary medium before heat absorbed by the first auxiliary medium is transferred to the methanol-containing stream by indirect heat transfer in the distillation evaporator.

In this preferred embodiment, the heat integration of the overall system is improved when the reactor arrangement has at least one water-cooled reactor. Typically, boiling boiler feed water is used as cooling medium for the indirect cooling of the catalyst bed in the methanol synthesis reactor in order to make use of the principle of evaporative cooling. The steam stream generated here on the shell side of the reactor, in particular a low-pressure steam stream, can be at least partly combined with the first auxiliary medium before the heat absorbed by the first auxiliary medium is transferred to the methanol-containing stream via indirect heat transfer in the distillation evaporator. In other words, the first auxiliary medium, after the transfer of heat from the heat pump end medium to the first auxiliary medium, is supplemented by the aforementioned steam stream, and then the heat is transferred from the first auxiliary medium to the methanol-containing stream in the distillation evaporator. Not only is heat absorbed from the heat pump medium by the first auxiliary medium transferred here, but also heat present in the steam stream.

A preferred embodiment of the process according to the invention is characterized in that waste heat generated in the electrolyser is transferred proximately to the heat pump medium by indirect heat transfer from an electrolysis medium.

This preferred embodiment corresponds to an example in which waste heat generated in the electrolyser is transferred proximately by indirect heat transfer to the heat pump medium, which is associated with the advantages elucidated above.

The waste heat generated in the electrolyser is especially stored in the electrolysis medium, which exits from the electrolysis cell stack of the electrolyser on completion of electrolysis reaction. Waste heat from this medium is transferred proximately or remotely by indirect heat transfer to the heat pump medium, as a result of which the latter is at least partly evaporated. The transfer of heat in the electrolysis medium, i.e. at least some of the waste heat from the electrolyser, may precede or follow the separation of product gases from the electrolysis medium, but should preferably be effected before the electrolysis medium that has been depleted of product gases re-enters the electrolysis cell stack.

A preferred embodiment of the process according to the invention is characterized in that waste heat generated in the electrolyser is transferred to a second auxiliary medium by indirect heat transfer, and heat absorbed by the second auxiliary medium is transferred to the heat pump medium.

This preferred embodiment corresponds to an example in which waste heat generated in the electrolyser is transferred remotely by indirect heat transfer to the heat pump medium. In this preferred embodiment, waste heat present in an electrolysis medium is first transferred indirectly to a second auxiliary medium, and then by indirect heat transfer from the second auxiliary medium to the heat pump medium.

A preferred embodiment of the process according to the invention is characterized in that the reactor arrangement includes a water-cooled reactor, and a steam stream is generated in a cooling system of the water-cooled reactor, and the steam stream is at least partly combined with the second auxiliary medium before heat absorbed by the second auxiliary medium is transferred to the heat pump medium.

In this preferred embodiment, the heat integration of the overall system is improved analogously to the above-described embodiment when the reactor arrangement has at least one water-cooled reactor. Here, however, in relation to the transfer of heat to the heat pump medium, this is effected such that the latter at least partly evaporates. In this embodiment, the steam stream generated on the shell side of a water-cooled reactor, especially a low-pressure steam stream, is at least partly combined with the second auxiliary medium before the waste heat absorbed from the electrolyser by the second auxiliary medium is transferred to the heat pump medium by indirect heat transfer, such that the latter at least partly evaporates. In other words, the second auxiliary medium is supplemented by the aforementioned steam stream, and then the heat is transferred from the second auxiliary medium to the heat pump medium. Not only is waste heat generated by the electrolyser transferred here to the heat pump medium, but also heat present in the steam stream.

A preferred embodiment of the process according to the invention is characterized in that the heat pump medium includes water, where a steam stream is utilized for the compression of the heat pump medium.

In particular, it is preferable that the reactor arrangement includes a water-cooled reactor, and a steam stream is generated in a cooling system of the water-cooled reactor and is used at least partly for the compression of the heat pump medium.

It is further preferable here that the heat pump system includes a jet pump for compression of the heat pump medium, where the jet pump has a suction port, a pressure port and a motive media connection, and where the jet pump is supplied via the suction port with the heat pump medium to be compressed, and is supplied via the motive media connection with the steam stream, and the compressed heat pump medium is discharged from the jet pump via the pressure port.

If water or at least one aqueous medium is utilized as heat pump medium, a vapour stream may be used for compression of at least a portion of the heat pump medium.

In that case, little electrical energy, if any, is required for compression of this portion of the heat pump medium, for example by means of a piston compressor or turbo compressor.

For this purpose, preference is given to utilizing at least a portion of the steam stream which is generated in the cooling system of a water-cooled reactor of the reactor arrangement.

Further preferably, the compression of at least a portion of the heat pump medium is effected by means of the steam stream in a jet pump. The jet pump may alternatively also be referred to as motive pump. Since the jet pump in this embodiment has a compressing effect, it can also be referred to as injector. The jet pump has a motive media connection, a suction port, and a pressure port. The steam stream is introduced into the jet pump via the motive media connection, and the expanded heat pump medium via the suction port. With the aid of the steam stream, the pressure of the heat pump medium in the jet pump is increased; the heat pump medium is thus compressed by the steam stream. Compressed heat pump medium is discharged from the jet pump via the pressure port. The pressure at the motive media connection is higher than the pressure at the pressure port, and the pressure at the pressure port is higher in turn than the pressure at the suction port. Since the amount of heat pump media is increased by the supply of steam at the motive media connection of the jet pump, a portion of the heat pump medium is preferably branched off from the heat pump media circuit upstream of the position of heat transfer to the liquid medium. Since this is condensed water in particular, it is preferably fed to the cooling system of the water-cooled reactor of the reactor arrangement as cooling water. This completes the circuit between water-cooled reactor and circulating heat pump medium.

If water or at least an aqueous medium is utilized as heat pump medium, the heat pump medium can be compressed by combination of a jet pump and a compressor. It is possible here to connect the jet pump and the compressor in series or in parallel.

For this purpose, preference is given to utilizing at least a portion of the steam stream which is generated in the cooling system of a water-cooled reactor of the reactor arrangement.

A preferred embodiment of the process according to the invention is characterized in that the heat pump system is designed as a high-temperature heat pump system in which the compressed heat pump medium is at a temperature of at least 60° C., preferably at a temperature of at least 100° C.

Further preferably, the heat pump medium has a temperature of not more than 200° C., preferably of not more than 180° C.

A preferred embodiment of the process according to the invention is characterized in that the carbon oxide component of the synthesis gas stream comprises at least 50 mol % of carbon dioxide, and at least a portion of the carbon oxide component is produced by a carbon dioxide separation process.

Further preferably, the carbon oxide component of the synthesis gas stream comprises at least 75% by volume of carbon dioxide, further preferably at least 90% by volume of carbon dioxide, further preferably at least 95% by volume of carbon dioxide, further preferably at least 99% by volume of carbon dioxide, further preferably at least 99.5% by volume of carbon dioxide.

Carbon dioxide deposition methods are understood here to mean a method that more or less selectively separates carbon dioxide from a gas mixture stream. The separation can be effected by a cryogenic method comprising condensation and optionally a cryogenic distillation of carbon dioxide. In addition, such a process may comprise one or more separation steps brought about by membranes. In addition, the separation may comprise an absorptive process comprising at least one physical or chemical absorption step, and a subsequent desorption step. In addition, the separation may comprise an adsorptive process comprising an adsorption step at a solid surface and a subsequent desorption step from that surface. In this case, the carbon dioxide itself need not necessarily be that species that binds adsorptively at the respective surface. In addition, the separation may comprise a membrane-based process that produces at least one retentate stream and one permeate stream, of which at least one stream has a higher carbon dioxide content than the feed stream supplied to the membrane.

### BRIEF DESCRIPTION OF THE FIGURES

The invention is elucidated in detail hereinafter by three working examples and one numerical example. The working examples are illustrated by FIGS. 1 to 3. In the figures, functionally and/or structurally identical or at least similar constituents are given identical reference numerals.

The Figures Show:

FIG. 1 a simplified block flow diagram of a first example of the process according to the invention,

FIG. 2 a simplified block flow diagram of a second example of the process according to the invention,

FIG. 3 a simplified block flow diagram of a third example of the process according to the invention.

### DETAILED DESCRIPTION OF THE INVENTION

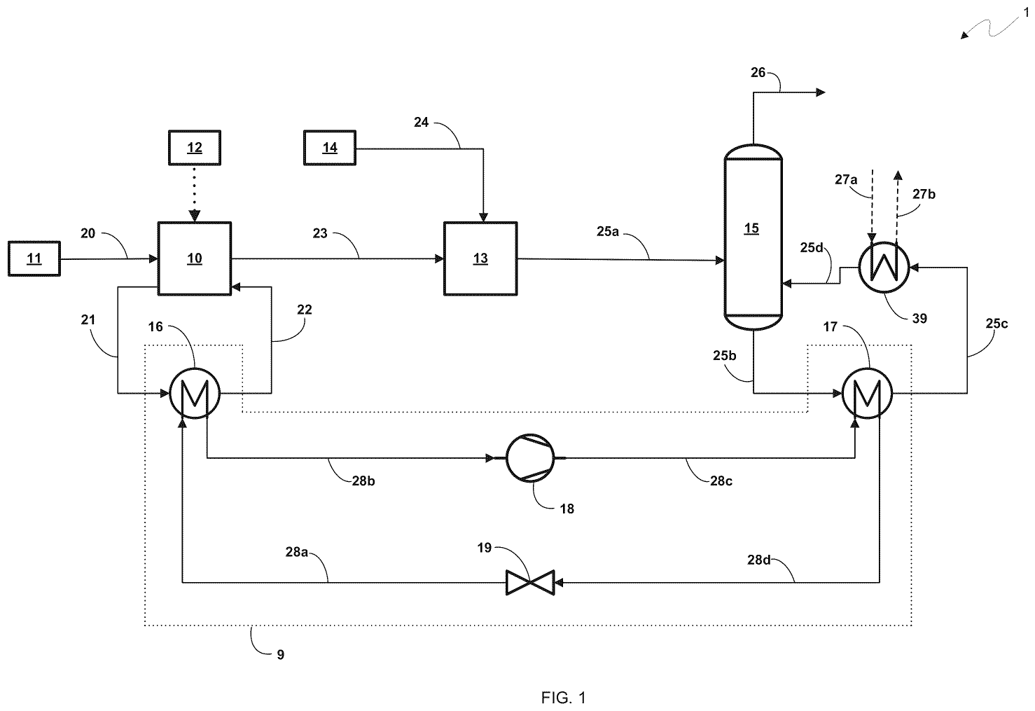

FIG. 1 shows a block flow diagram of a process 1 according to a first example of the invention.

In the example according to FIG. 1, both the transfer of the waste heat from an electrolyser to the heat pump medium and the transfer of the heat of condensation of the heat pump medium to the methanol-containing stream are effected proximately by indirect heat transfer, i.e. without auxiliary heat transfer medium.

An electrolyser 10, for example a PEM electrolyser, is supplied with a water stream 20 via a water reservoir 11, and with electrical energy via a DC power source 12. By electrochemical cleavage of water as electrolysis medium, the electrolyser produces an oxygen stream (not shown) and a hydrogen stream 23. The oxygen stream is released to the atmosphere. Alternatively, it would also be possible to release the oxygen stream to another process or to another point in the methanol plant. The hydrogen stream 23 is fed to a methanol synthesis reactor arrangement 13 comprising at least a water-cooled methanol synthesis reactor, a cooler, a condenser, and a separator (details not shown). The methanol synthesis reactor arrangement 13 is also supplied with a carbon dioxide stream 24 from a carbon dioxide source 14. The carbon dioxide source 14 is the regeneration column of an amine scrubbing unit. In the methanol synthesis reactor arrangement 13, hydrogen in the hydrogen stream and carbon dioxide in the carbon dioxide stream 24 are converted over a methanol synthesis catalyst to a crude methanol stream. The crude methanol stream containing at least methanol and water is first fed to a primary column in which low-boiling components are removed as top product (not shown). The bottom product from the primary column is fed as methanol-containing feed stream 25a to a distillation column 15 in which a methanol stream 26 is produced as top product, as is a water stream (not shown) as bottom product.

The process also includes a heat pump system 9 comprising a heat exchanger 16, a heat exchanger 17, a compressor 18, and a throttle valve 19. The aforementioned components are fluidically connected to one another by corresponding conduits, such that a heat pump medium can circulate between the components mentioned. The heat exchanger 16 has the function of the evaporator of the heat pump system, and the heat exchanger 17 the function of the liquefier.

A stream of a hot electrolysis medium 21 is discharged from the electrolyser 10, the heat from which is partly transferred in the heat exchanger 16 to a liquid heat pump medium 28a entering said heat exchanger 16. The heat pump medium 28a is evaporated as a result. The hot electrolysis medium 21 is correspondingly cooled, as a result of which a stream of a cooled electrolysis medium 22 is discharged from the heat exchanger 16 and returned to the electrolyser. The evaporated heat pump medium 28b is fed to the compressor 18, which increases the temperature and pressure of the evaporated heat pump medium. The compressed heat pump medium 28c is fed to the heat exchanger 17, in which the heat pump medium is liquefied (condensed) in heat exchange with the methanol-containing stream 25b drawn off from the distillation column 15. The heat exchanger 17 in this case forms part not only of the heat pump system 9, but also part of the distillation evaporator of the distillation column 15. In the heat exchanger 17, the methanol-containing stream 25b is partly, but not completely, evaporated by the transfer of the heat of condensation from the heat pump medium. For complete evaporation, the partly evaporated methanol-containing stream 25c is fed to a further heat exchanger 39, which likewise forms part of the distillation evaporator of the distillation column 15. The heat exchanger 39 is supplied with a steam stream 27a which completely evaporates the methanol-containing stream 25c. The completely evaporated methanol-containing stream 25d enters distillation column 15 in order to be separated thermally therein into a methanol stream 26 and a water stream (not shown). The vapour stream 27a condenses in the heat exchanger 39, and leaves the heat exchanger 39 as condensate stream 27b.

The stream of the condensed, cooled heat pump medium 28d is expanded by the throttle valve 19 and fed as expanded heat pump media stream 28a to the heat exchanger 16 (evaporator), in which the heat pump medium is evaporated again by absorption of the waste heat from the electrolyser 10.

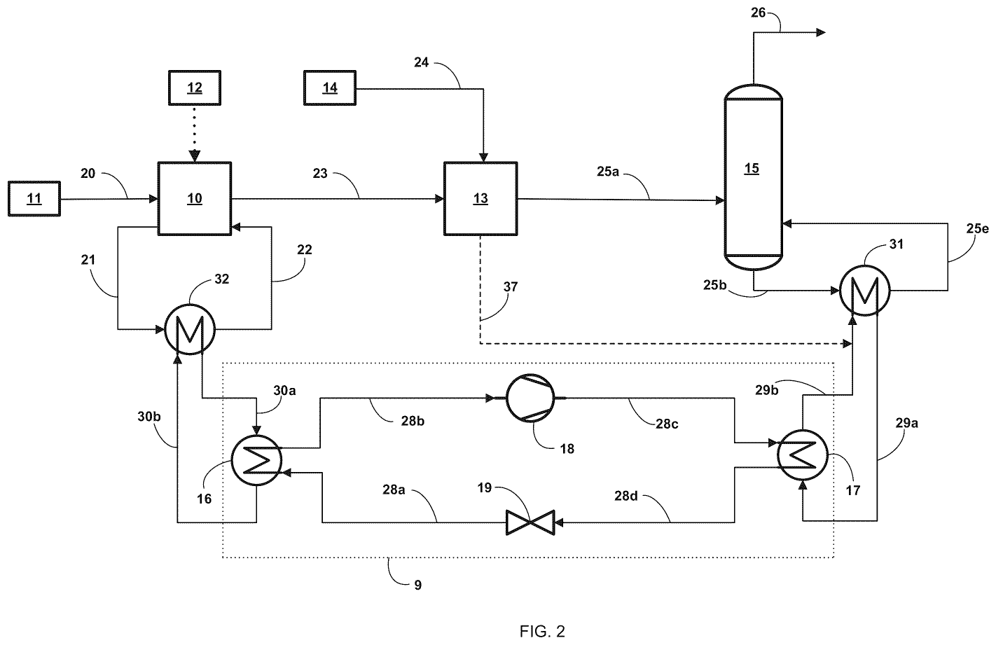

FIG. 2 shows a block flow diagram of a process 2 according to a second example of the invention.

In the example according to FIG. 2, both the transfer of the waste heat from the electrolyser to the heat pump medium and the transfer of the heat of condensation of the heat pump medium to the methanol-containing stream are effected remotely by indirect heat transfer, i.e. in each case utilizing an auxiliary heat transfer medium with intermediate heat exchangers.

What are addressed hereinafter are essentially differences from the example according to FIG. 1.

The waste heat from the electrolyser 10 is first transferred in a heat exchanger 32, which is supplied with a stream of a cooled second auxiliary medium 30b. Heat from the stream of the hot electrolysis medium 21 is transferred in the heat exchanger 32 to the stream of the cooled auxiliary medium 30b. The stream of auxiliary medium 30a that has been heated as a result is discharged from the heat exchanger 32 and, in the heat exchanger 16 (evaporator), has the effect of evaporating the heat pump medium 28a supplied to said heat exchanger, which is then fed as evaporated heat pump medium 28b to the compressor 18.

On the liquefier side of the heat pump system 9, a cooled stream of a first auxiliary medium 29a has the effect of condensing the compressed heat pump medium stream 28c. The resulting heated auxiliary media stream 29b is combined with a steam stream 37, which is withdrawn from the cooling system of a water-cooled reactor of the methanol synthesis reactor arrangement 13. A correspondingly combined stream of steam and heated first auxiliary medium is fed to a heat exchanger 31 in which the evaporation of the methanol-containing stream 25b is brought about. The evaporated methanol-containing stream 25a is fed to the distillation column 15 for thermal separation into the methanol stream 26 and water stream (not shown). The heat exchanger 31 here is the distillation evaporator of distillation column 15 and does not form part of the heat pump system 9.

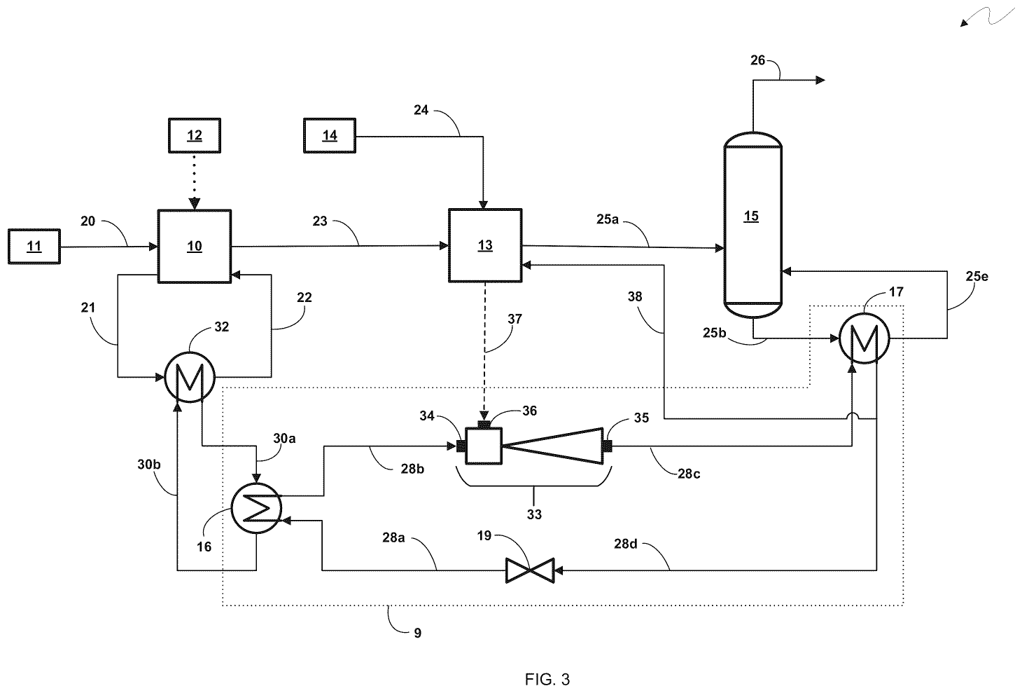

FIG. 3 shows a block flow diagram of a process 3 according to a third example of the invention.

In the example according to FIG. 3, the waste heat from the electrolyser is transferred analogously to the example from FIG. 2 with the aid of a second auxiliary medium, firstly in the heat exchanger 32 to the second auxiliary medium, and then in the heat exchanger 16 from the second auxiliary medium to the expanded heat pump medium 28a, which is evaporated as a result.

In the example according to FIG. 3, the heat pump medium used is water.

The stream of the evaporated heat pump medium 28b is fed via a suction port 34 to a jet pump 33. The jet pump is also supplied with a steam stream 37 via a motive media connection 36. The steam stream 37 is withdrawn from the cooling system of a water-cooled reactor of the methanol synthesis reactor arrangement 13. The steam stream 37 compresses the vaporous heat pump medium 28b in the jet pump 33, and is discharged from the jet pump 33 as a stream of a compressed heat pump medium 28c via a pressure port 35 thereof. The compressed heat pump medium 28c is condensed in the heat exchanger 17 and hence heats the methanol-containing stream 25b, which is evaporated as a result and enters the distillation column 15 as gaseous methanol-containing stream 25e.

A substream 38 is branched off from the stream of the condensed heat pump medium 28d for the purpose of mass balance compensation. This substream 38 is fed to the cooling system of the water-cooled reactor of the methanol synthesis reactor arrangement 13 for reuse as cooling water.

The numerical example which follows, which is based on simulation data, is also intended to illustrate the advantages of the process according to the invention.

A typical 100 MW PEM electrolyser under full load generates an amount of heat of about 20 to 25 MW at about 50° C. A methanol plant that produces methanol at a rate of about 220 t of methanol per day under full load on the basis of carbon dioxide and electrolytically produced hydrogen has a heat import requirement of at least around 10 MW at at least around 130° C.

According to the example that follows, the electrolyser transfers its waste heat proximately by means of a heat exchanger to the heat pump medium of the heat pump system, and the compressed and heated heat pump medium transfers its heat proximately by means of a heat exchanger to liquid crude methanol in the distillation evaporator.

The waste heat from the electrolyser can be transferred in the following manner:
10 MW of waste heat from the process medium of the electrolyser (electrolysis medium at 60° C.) is transferred to the heat pump medium, with evaporation of the latter, which has a temperature of 50° C. after evaporation.
The evaporated heat pump medium is fed to a compressor, which increases the temperature of the heat pump medium to 130° C. and increases the pressure of the heat pump medium. Taking account of known high-temperature heat pump curves, compression of the heat pump medium containing 10 MW requires about 3.3 MW of power (for example in the form of electricity). The figure of merit is thus 3.
The heat pump medium at 130° C. condenses on the hot side of the methanol distillation column and provides 10 MW in the heat exchanger of the distillation evaporator via the heat of condensation.

For comparison: If saturated steam at 130° C. is to be produced at 2.7 bar from water by supply of electrical energy, this would consume the entire 10 MW transferred in the heat exchanger of the distillation evaporator.

Savings resulting from the use of the present invention in this case are 10 MW (full power consumption for the heating for generation of saturated steam) minus 3.3 MW (power consumption of the heat pump system), which gives a saving of 6.7 MW. This power is “saved”, which means that it is not consumed in the form of additional power, but can be provided via utilization of the waste heat from the electrolyser.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

### LIST OF REFERENCE NUMERALS

1, 2, 3 process
9 heat pump system
10 electrolyser
11 water reservoir
12 DC power source
13 methanol synthesis reactor arrangement
14 carbon dioxide source
15 distillation column
16 heat exchanger (evaporator)
17 heat exchanger (liquefier)
18 compressor
19 throttle valve
20 water stream
21 hot electrolysis medium
22 cooled electrolysis medium
23 hydrogen stream
24 carbon dioxide stream
25a methanol-containing feed stream
25b, 25c, 25d, 25e methanol-containing stream
26 methanol stream
27a steam stream
227b condensate stream
28a, 28b, 28c, 28d circulating heat pump medium
29a, 29b stream of the first auxiliary medium
30a, 30b stream of the second auxiliary medium
31 heat exchanger for first auxiliary medium
32 heat exchanger for second auxiliary medium
33 jet pump
34 suction port
35 pressure port
36 motive media connection
37 steam stream
38 condensate stream
39 heat exchanger

The invention claimed is:

1. A process for producing methanol, comprising:
a) providing a synthesis gas stream comprising at least a hydrogen component and a carbon oxide component, with at least one of the components being provided at least partly by an electrolyser;
b) converting the synthesis gas stream over a methanol synthesis catalyst in a reactor arrangement to give a gaseous crude methanol stream;
c) producing a liquid crude methanol stream by condensing and separating the crude methanol stream obtained in step b);
d) thermally separating methanol from the liquid crude methanol stream in a distillation apparatus, where the distillation apparatus has at least one distillation column in which a methanol-containing stream is evaporated in a distillation evaporator,
wherein, waste heat generated in the electrolyser is transferred by indirect heat transfer to a circulating heat pump medium of a heat pump system, which results in at least partial evaporation of the heat pump medium, then the heat pump medium is compressed, and the heat released by the heat pump medium via at least partial condensation is transferred to a liquid medium by indirect heat transfer, and the heat transferred from the heat pump system to the liquid medium is utilized at least partly for the evaporation of the methanol-containing stream in the distillation evaporator in step d).

2. The process of claim 1, wherein the heat released by the heat pump medium is transferred proximately to the methanol-containing stream by indirect heat transfer in the distillation evaporator, where the methanol-containing stream comprises the liquid medium.

3. The process of claim 1, wherein the heat released by the heat pump medium is transferred to a first auxiliary medium by indirect heat transfer, where the first auxiliary medium comprises the liquid medium, and heat absorbed by the first auxiliary medium is transferred to the methanol-containing stream by indirect heat transfer in the distillation evaporator.

4. The process of claim 3, wherein the reactor arrangement includes a water-cooled reactor, and a steam stream is generated in a cooling system of the water-cooled reactor, and the steam stream is at least partly combined with the first auxiliary medium before heat absorbed by the first auxiliary medium is transferred to the methanol-containing stream by indirect heat transfer in the distillation evaporator.

5. The process of claim 1, wherein the waste heat generated in the electrolyser is transferred proximately to the heat pump medium by indirect heat transfer from an electrolysis medium.

6. The process of claim 1, wherein the waste heat generated in the electrolyser is transferred to a second auxiliary medium by indirect heat transfer, and heat absorbed by the second auxiliary medium is transferred to the heat pump medium.

7. The process of claim 6, wherein the reactor arrangement includes a water-cooled reactor, and a steam stream is generated in a cooling system of the water-cooled reactor, and the steam stream is at least partly combined with the second auxiliary medium before heat absorbed by the second auxiliary medium is transferred to the heat pump medium.

8. The process of claim 1, wherein the heat pump medium includes water, where a steam stream is used for the compression of the heat pump medium.

9. The process of claim 8, wherein the reactor arrangement includes a water-cooled reactor, and a steam stream is generated in a cooling system of the water-cooled reactor and is used at least partly for the compression of the heat pump medium.

10. The process of claim 8, wherein the heat pump system includes a jet pump for compression of the heat pump medium, where the jet pump has a suction port, a pressure port and a motive media connection, and where the jet pump is supplied via the suction port with the heat pump medium to be compressed, and is supplied via the motive media connection with the steam stream, and the compressed heat pump medium is discharged from the jet pump via the pressure port.

11. The process of claim 1, wherein the heat pump system is designed as a high-temperature heat pump system in which the compressed heat pump medium is at a temperature of at least 60° C.

12. The process of claim 1, wherein the carbon oxide component of the synthesis gas stream comprises at least 50 mol % of carbon dioxide, and at least a portion of the carbon oxide component is produced by a carbon dioxide separation process.
